(19) United States
(12) Patent Application Publication
(10) Pub. No.: US 2012/0164636 A1
(43) Pub. Date: Jun. 28, 2012

(54) MAMMALIAN OOCYTE DEVELOPMENT COMPETENCY GRANULOSA MARKERS AND USES THEREOF

(75) Inventors: Marc-Andre Sirard, Breakeyville (CA); Melanie Hamel, St-Augustin-de-Desmaures (CA); Claude Robert, Saint-Nicolas (CA)

(73) Assignee: UNIVERSITE LAVAL, Quebec, QC (CA)

(21) Appl. No.: 13/413,566

(22) Filed: Mar. 6, 2012

Related U.S. Application Data
(63) Continuation of application No. 12/441,278, filed on Sep. 25, 2009.
(60) Provisional application No. 60/825,814, filed on Sep. 15, 2006.

(51) Int. Cl. C12Q 1/68; C12Q 1/02

(52) U.S. Cl. 435/6.1; 435/29

(57) ABSTRACT

The present invention relates to the competence of oocytes to uterine implantation and development into living individual. The invention more particularly relates to marker that are detected and measured in granulosa cells collected along with the oocytes during oocyte aspiration as it is done in assisted reproduction techniques.

### CROSS REFERENCE TO RELATED APPLICATIONS

The present application is a continuation of U.S. Non-Provisional application Ser. No. 12/441,278, filed Mar. 13, 2009, which is a national phase application under 35 U.S.C. 371 of International Application No. PCT/CA07/01633, filed on Sep. 14, 2007, which claims priority to U.S. Provisional Application No. 60/825,814, filed on Sep. 15, 2006. The entirety of each of these priority applications is incorporated herein by reference.

### BACKGROUND OF THE INVENTION

a) Field of the Invention

The present invention relates to granulosa markers of mammalian oocyte competency to develop into healthy fetuses and live born babies and uses thereof.

b) Description of the Prior Art

Oocyte's quality largely depends on the follicle from which it originates, as shown in a number of animal and human studies. During the IVF procedure upon ovarian stimulation and ovulation induction, a cohort of heterogeneous follicles is recruited to develop and ovulate, irrespective of their differentiate state. This creates an asynchrony in the maturation process and heterogeneity in the quality of the oocytes recovered for assisted reproduction. To determine the factors associated with the developmental competence of the oocytes and to understand how they positively influence the oocyte quality, follicles with different oocyte quality must be analyzed for these factors at the protein and gene levels.

Previous studies have tended to focus upon the appearance of the embryo (morphology) to predict the success of fertilization in vitro. Other means of investigate the embryo quality may interfere with embryo viability leading to an absence of objective criteria to distinguish between several embryos, which to transfer to the mother. In recent years, scientific evidences obtained both from animal models and humans are supporting the hypothesis that the oocyte quality and therefore its ability to implant post transfer depends on the follicular conditions prevailing in the ovary before the oocytes are removed. This leads to a method of predicting the outcome of IVF which involved firstly determining the level of target compounds in a biological sample taken from a female patient and then predicting, from the level of the compounds determined, the probability of establishing pregnancy in the subject by IVF. The activity measured for a pool of cells from different follicles (from the same individual) was not always a true reflection of activity in individual follicles, suggesting that one or more follicles possess compounds affecting the probability of establishing a pregnancy

A major problem in identifying which oocytes are competent to become embryos is the fact that any procedure designed for such purpose must not adversely affect the quality or viability of the oocytes.

US Patent publication no. 20060147900 describes cumulus specific markers (e.g., pentraxin 3), and methods of using the same to determine oocyte development potential. Limited number of markers are described. It is shown in the art (Garlanda et al., J. Soc. Gyneclo. Investig., 2006: 13: 226-231) that elevated levels of soluble pentraxin 3 can be found in follicular fluid, that follicular fluid concentration of pentraxin 3 cannot be used as a marker of oocyte quality, and that plasma concentration of the pentraxin 3 is not influenced by ovarian hyperstimulation. Also, pentraxin 3 gene expression was not detected in granulosa cells (Matzuk et al., 2004, Human Reproduction, 19:2869-2874).

Considering the state of the art, there is still needs for markers for determining the competency of oocytes for uterus implantation and development in a living individual.

### SUMMARY OF THE INVENTION

In accordance with one embodiment of the present invention, there is provided a granulosa cell marker for determining competence of an oocyte from a patient for in vitro fertilization (IVF), uterus implantation and/or development in a living individual at birth, which comprises at least one polynucleotide or polypeptide chosen from CYP19A1, CDC42, DPYSL3, 3βHSD1, EREG, SERPINE2, SCARB1, INHBA, SPRY 2, BACH2, ILST6, ADX, TNFAIP6, SERPINA3, EGR1, NRP1, RGS2, and PGK1, full-length cDNA clones and combinations thereof.

The oocyte may be from a mammal. The oocyte and granulosa cell marker may be from a single follicle. The polynucleotide may be a DNA or a RNA sequence.

In accordance with another embodiment of the present invention, there is provided a method for determining competence of an oocyte from a patient for IVF, uterus implantation and/or development in a living individual at birth, said method comprising determining expression level of a granulosa cell marker from granulosa cells obtained from said patient, wherein said marker comprises at least one polynucleotide or polypeptide chosen from CYP19A1, CDC42, DPYSL3, 3βHSD1, EREG, SERPINE2, SCARB1, INHBA, SPRY 2, BACH2, ILST6, ADX, TNFAIP6, SERPINA3, EGR1, NRP1, RGS2, nad PGK1, full-length cDNA clones and combinations thereof, and wherein expression level of said marker from a granulose cell of an oocyte that is higher than the expression level of said marker of a control granulosa cell from said follicle is representative of competency of said oocyte to uterus implantation and development in a living individual.

The patient may be a mammal. The oocyte and said granulosa cells may be from a single follicle.

The method may further comprises comparing the expression level with expression level of control granulosa cells and showing a significant change by using ratios or absolute amount to reflect oocyte competence.

In accordance with the method of the present invention, the granulosa cell may be obtained by aspiration of follicular fluid before ovulation.

In accordance with the method of the present invention, the expression levels of ADX, CYP19A1, CDC42, SERPINE2, and 3βHSD1 are determined.

In accordance with the method of the present invention, the expression levels of at least two markers chosen from CYP19A1, CDC42, DPYSL3, 3βHSD1, EREG, SERPINE2, SCARB1, INHBA, SPRY 2, BACH2, ILST6, ADX, TNFAIP6, SERPINA3, EGR1, NRP1, RGS2, and PGK1 are determined.

In accordance with the method of the present invention, the expression levels of at least three markers chosen from CYP19A1, CDC42, DPYSL3, 3βHSD1, EREG, SERPINE2, SCARB1, INHBA, SPRY 2, BACH2, ILST6, ADX, TNFAIP6, SERPINA3, EGR1, NRP1, RGS2, and PGK1 are determined.

In accordance with another embodiment of the present invention, there is provided a method for screening a compound stimulatory or inhibitory to oocyte competence to IVF, uterus implantation or development into living individual at birth, said method comprising the steps of;

a) treating granulosa cells with a compound to be screened for activity to stimulate or inhibit the competence of an oocyte to IVF, uterus implantation or development into living individual at birth;

b) determining the expression level of at least one marker as defined in claim 1 in said granulosa cells;

c) comparing the expression level measured in step b) with the expression level of control granulosa cells reflecting oocyte competence.

The ratio of expression level of a marker in treated granulosa cells over the expression level of a marker in control granulosa cells higher than 1.5 is indicative of stimulatory effect of said compound in expression of said markers, and said ratio being lower than 1 is indicative of inhibitory effect.

The treatment is performed in vitro or in vivo.

For the purpose of the present invention the following terms are defined below.

The term “patient” is intended to mean an animal or a mammal, including, but not limited to, human, primate, bovine, porcine, caprine, rodent, ungulates, vertebrates, equines, felines, ayes, ruminants, among others, from which the oocyte competence is tested according to the present invention, and to which a treatment can be applied. The term patient can be alternatively identified by the terms subject or individual, which are used herein interchangeably by meaning the same thing

The term “recipient” as used herein is intended to mean a human or an animal female into which a fertilized oocyte, or embryo, tested according to the present invention, is transferred. The person skilled in the art will recognize that the oocyte can be obtained at a desired stage by in vivo or in vitro maturation, as well as the embryo can be produced by in vitro fertilization or sperm nuclear transfer into the oocyte.

The term “competence” as used herein is intended to mean the competence, or competency, both terms being equivalent, of an oocyte for implantation and development into living individual. The subject matter of the present invention provides predictor values for determining the competency of an oocyte also in selecting embryos to be transferred to a recipient.

The expression “granulosa cells” as used herein defines follicular mural cells. When the antrum develops and enlarges, the granulosa cells divide into two functional groups: the cells in immediate contact with the oocyte which are called the cumulus cells (cumulus oophorus) and the mural granulosa cells which line the follicular wall around the antrum.

Cumulus cells express characteristics distinct from the mural granulosa cells.

### BRIEF DESCRIPTION OF THE DRAWINGS

FIG. 1 illustrates Venn diagram of the four hybridizations for pools 1 and 2 with the custom-made granulosa microarray (2,278 transcripts) and the Affymetrix human U133 GeneChip® (44,700 transcripts). Numbers represent the number of different genes showing a signal above the background threshold. Numbers in parenthesis represent the number of different transcripts showing a signal above the background threshold.

FIG. 2 illustrates the quantification of mRNA level by real-time PCR that showed differential expression (P<0.05) in granulosa cells from follicles that resulted in a pregnancy (Positive groups) and between granulosa cells from follicles that produced embryos that arrested in development (Negative groups). ** Indicates a significant difference within gene (P<0.01), * Indicates a significant difference within gene (P<0.05). Results were presented as mean±SEM and analyzed by t-test analysis.

FIG. 3 illustrates the quantification of mRNA level by real-time PCR with no significant differential expression (P<0.05) but showed a tendency to be expressed differentially in granulosa cells from follicles that resulted in a pregnancy (Positive groups) and between granulosa cells form follicles that produced embryos that arrested in development (Negative groups). Results are presented as mean±SEM and analyzed by t-test analysis.

### DESCRIPTION OF THE PREFERRED EMBODIMENT

The present invention now will be described more fully hereinafter with reference to the accompanying drawings, in which preferred embodiments of the invention are shown. This invention, may, however, be embodied in many different forms and should not be construed as limited to the embodiments set forth herein; rather, these embodiments are provided so that this disclosure will be thorough and complete, and will fully convey the scope of the invention to those skilled in the art.

In accordance with the present invention, there is provided polypeptides and nucleotide sequences encoding mRNA or the polypeptides which allow, through their measurement in granulosa cells to predict the competency of an oocyte from the same follicle from which are measured the granulosa cells, to implantation and development into living individual (or, more accurately, successful implantation), and in particular to predict the outcome of in vitro fertilization (IVF) and implantation in a female individual.

The invention also relates to such methods of diagnosis in order to determine the outcome of IVF or the suitability of a female individual for assisted reproduction treatment. Oocytes control their environment by suppressing differentiation of the mural granulosa cell phenotype and promoting differentiation of the cumulus cell phenotype. They achieve this suppression via the secretion of labile paracrine signaling factors. Errors in this regulatory mechanism, whether instigated by defects in the production of oocyte-derived ligands or granulosa cell responses to them, may result in the production of oocytes unable to undergo embryo development or that undergo abnormal follicular development.

According to the present invention, mural granulosa cells, or granulosa cells, can be harvested directly by aspiration, as known in the art, through the follicle in the patient's vagina with an appropriate needle. This context defines the oocytes as in vivo oocytes. Granulosa cells can also be obtained by their puncture from an ovary outside the patient's body.

In another embodiment of the present invention, there is provided granulosa specific markers, polypeptides and nucleotide sequences encoding thereof, for determining the competence of fertilized oocytes, or embryos, to implant in the uterus of a recipient female, and to develop into a living individual. The markers and their use are therefore useful to perform the screening of competent embryos before their transfer in a recipient human or animal female.

In another embodiment of the present invention, there is provided markers selected from the group consisting of Fas oncogene, Fas ligand (FasL), Bax, inhibitor of apoptosis X (XIAP), NIAP, HIAP-1, HIAP-2, BCLxI, FLIP, PAL31, bone morphogenic protein 15 (BMP-15), caspase cleavage or activation protein 3, 7, 8, or 9, AkT phosphorylation protein, DNA binding protein A, matrix metalloproteinase (MMP), ribosomal L27a, Sprouty 2, early growth response 1, (Erg-1), phosphatidylserine synthase 1, cytochrome C oxidase, metalloproteinase (MMP) inducer, protein kinase inhibitor, reverse transcriptase, versicant V3 variant, ring finger protein 13, acidic ribosomal protein PO, prostaglandin receptor (EP3B), progesterone receptor, epiregulin (ERG), splicing factor, and transglutaminase 3, measured in granulosa cells for determining the capacity, or competence, of an oocyte originating from the same follicle from which are obtained the granulosa cells, to IVF, uterine implantation, and development into living baby. The expression level of nucleic acid sequences can be also determined to assess the competence of a tested oocyte.

In the case where the expression level of a marker in granulosa cells of a tested oocyte is in the range associated with competent follicles (compared to the range of follicles leading to incompetent oocytes) the tested oocyte will be deemed competent for the implantation in the uterine wall of a female, and to develop into a living individual, as well as a fetus as a live born baby. This results in a better predictability to have successful pregnancy and healthy baby from a selected oocyte and embryo. The average expression level of target markers, under the form of polypeptides or nucleotides, which is representative of the competence of the oocyte as defined herein, is used to select or to assess oocytes likely to implant and to develop properly in the uterus up until the birth.

According to another embodiment of the present invention, the expression level of markers is determined through the measurement of the marker polypeptides or their corresponding mRNA in the granulosa cells at the time the granulosa cells are taken by aspiration from the follicle with its oocyte. Any suitable method known in the art can be used to measure the marker's gene expression. Suitable measurement methods include, but not limited to, the use of nucleic acid probes capable of specifically hybridizing to the mRNA of interest, oligonucleotides or PCR primers capable of specifically amplifying the target nucleotide sequence, and antibodies capable of specifically binding to polypeptides expressed by the gene of interest. The gene expression includes, but is not limited to, the conversion of genetic information encoded in a gene into RNA, such as mRNA, rRNA, tRNA, or snRNA, through transcription of the gene by RNA transcriptase, and translation of the RNA into proteins or polypeptides corresponding to the gene expressed. Depending on the context the invention is carried out, the nucleic acid probes, oligonucleotides or PCR primers may be of about 5 to 200 nucleic acids in length. The ways of preparing such nucleic acid probes, oligonucleotides or PCR primers are well known by persons skilled in the art. PCR analysis is preferably performed as reverse-transcriptase PCT (RT-PCR). The reverse transcriptase convert RNA molecules into DNA fragments that can be amplified by PCR or T7 polymerase. The PCR amplification product can then be migrated on a gel electrophoresis to be visualized or measured in real time for precise quantification (Real-time PCR). For better results, the PCR primers can be themselves marked with stains, of radioactive nucleic acids.

The nucleic acid probe, PCR primers, or the like, includes, but not limited to, DNA or RNA, into which can be inserted for detection needs any known base analogs of DNA or RNA, or markers molecules, such as in case of, but not limited to, hybridization or amplification. Other methods, such as microarray analysis, Norther blot, Southern blot, or real-time PCR.

Alternatively, antibodies can be used to perform immunochemistry, ELISA (enzyme-linked immunosorbant assay), sandwich immunoassays, immunofluorometry, immunoradiometric assays, gel diffusion precipitation reactions, immunodiffusion assays, in situ immunoassays, Western blot, radioimmunoassay (RIA), a bioanalytical method that uses specific antibodies, or fragments thereof, and radiolabeled detector molecules to quantify a defined analyte in mixtures, or any other known method in the art using antibody to target a specific molecule. Many immunoassays can be performed using dyes or other markers in lieu of the radioactive label. Antibodies, or immunoglobulins, are proteins that recognize and bind specifically to an antigen, or an epitope. An epitope is the part of an antigen which bind to a specific antibody. The specificity degree of an antibody through an epitope is defined by its capability to bind only, or not, to its target epitope, or antigen. Antibodies include, but not limited to, polyclonal, monoclonal, chimeric, humanized antibodies, and Fab fragments. Are also included single chain and double chain antibodies. Antibodies can also be used for in vivo imaging detection as known in the art. This permits the detection of targeted markers directly into the body of a woman or animal female and predict if this woman or animal female has oocytes competent to fertilization, implantation and uterine development. Whether markers of the present invention are detected according to desired ratios, the tested woman or animal female can be considered as being competent to be fertilized and to become properly pregnant.

In another embodiment of the present invention, the competence of an oocyte can be addressed by the measurement of the expression level of one expression profile. The later allows to draw a gene expression profile pattern of a tested oocyte, this expression profile giving the possibility of establishing more finely the competence of an oocyte as defined herein.

In case the expression level of a marker in a tested oocyte is lower than the average level of the same marker in a group of competent oocytes, it is deemed not likely competent to become fertilized or to implant. On the contrary, a tested oocyte having an expression level of a marker similar to the controls (competent group) will be classified as being competent to IVF, implantation and intra-uterine development. The ratio of the expression level of a marker in a tested oocyte on the expression level of a marker in a control oocyte can be from about 1.5 above control to 150, and preferably above 2, for an oocyte to be deemed competent to IVF, implantation and uterine development into living baby.

In one embodiment, there is provided panels and kits for the detection of markers. The presence of a tested oocyte competence marker is used to determine the likelihood of the tested oocyte to properly allow IVF, or to implant into the uterus following transfer. The panels and kits can be used for simultaneous analysis of several markers, and to provide results giving gene expression profiles.

### Drug Screening

In another embodiment of the present invention, there is provided a method for screening candidate compounds capable of increasing or decreasing the expression of markers of the invention as described herein. For example, but not limited to, isolated granulosa cells put in in vitro culture conditions can be submitted to treatment with some compounds, and then tested for measuring the increase or decrease of gene expression levels of oocyte competence markers, therefore reflecting the compound effect. This approach will allow the screening of compounds stimulatory or inhibitory to oocyte competence. The same compound testing can be performed in in vivo conditions, that is to say administration of compounds to a woman or animal patient, through which ovarian stimulation conditions can be tested for the production of competent oocytes as defined herein.

### Competence Induction

In another embodiment of the present invention, there is provided a method for rendering an oocyte deemed non competent in an oocyte competent to IVF, implantation and uterine development. The method includes treating a non competent oocyte with a factor known to stimulate the expression of an oocyte-competence marker, as defined herein above. Measurement of the markers of the present invention then indicate if the stimulation has been efficient. Alternatively, the markers and method of use thereof of the present invention can be used to evaluate the responsiveness of a woman or animal female to an hormone treatment. By this embodiment, the use of luteinising hormone (LH) or human choriogonadotropin (hCG), or decreasing the concentration of follicle stimulating hormone (FSH), are examples, without limitation to, of how a hormonal treatment can enhance the oocyte competence, and its level of competence markers.

The present invention will be more readily understood by referring to the following examples which are given to illustrate the invention rather than to limit its scope.

### Example 1

### Markers in Human Follicular Cells Associated with Competent Oocytes

### Follicular Cells Collection

Follicular cells were obtained from women (n=40) that were undergoing IVF treatment with their consent at the Fertility Center at the Ottawa Hospital, Canada. These women had endometriosis, tubal or idiopathic infertility diagnosis but not polycystic ovary syndrome (PCO). The procedure was performed with approval from the Ottawa Hospital research ethics board. Following ovarian stimulation, follicular fluid, follicular cells and oocytes from individual follicles were collected by ultrasound-guided follicular aspiration using a double lumen needle. The oocytes and surrounding cumulus cells were removed for IVF treatment. The remaining follicular fluid was centrifuged at 800×g for 10 minutes at room temperature to isolate the follicular cells containing mural granulosa cells, for each individual follicle. The resulting pellet was suspended in 500 μl of phosphate buffered saline solution (PBS) at 4° C. and was transferred into a cryovial. After centrifugation at 2000×g for 1 minute at room temperature, the supernatant was removed and cells were rapidly frozen and stored in liquid nitrogen until RNA extraction. After the fertilization process, cumulus cells surrounding the oocytes were also recovered on an individual follicle basis using the same protocol as described for follicular cell isolation.

A range of 1 to 15 follicles were aspirated for an average of 7.48 follicles per patient, and an average of 4.13 embryos was obtained per woman. Data (fertilization, embryo development, embryo morphology, transfer and pregnancy) generated from each follicle was recorded by an embryologist. Depending of the IVF protocol used, one or two (average of 1.4) embryos were transferred at either day 3 (67%) or day 5 (33%) for a total of 34 patients with an overall per transfer pregnancy rate of 53%. Pregnancy was confirmed by the presence of a foetal heartbeat by ultrasound at 6 to 8 weeks.

### Treatment Assignment (Table 1)

For the mural granulosa cells, three pools of follicles [pool 1 (n=6), pool 2 (n=15) and pool 3 (n=9)] were created from oocytes that resulted in a successful pregnancy, which were called the Positive groups 1, 2 and 3 respectively. These pools were used to generate RNA associated with competent follicles. Three more pools [pool 1 (n=6), pool 2 (n=15) and pool 3 (n=9)] were assigned to the Negative groups 1, 2 and 3 respectively containing follicles resulting in embryos that arrested their development before the 8 cell stage and include one embryo that did not implant (group 1). Pool 1 was used to make the custom-made cDNA microarray, Pools 1 and 2 from both Positive and Negative groups served for array hybridizations while all 3 pools served for Q-PCR analysis. Cumulus cells from the same follicles selected for both the positive and negative group 1 were used separately to make the custom-made cDNA microarray.

TABLE 1
Treatment assignment

| | POOL 1 | POOL 2 | POOL 3 |
|---|---|---|---|
| Positive | Transferred | Transferred | Transferred |
| Groups | oocyte with | oocyte with | oocyte with |
| | pregnancy | pregnancy | pregnancy |
| | 6 follicles | 15 follicles | 9 follicles |
| | 4 patients | 9 patients | 5 patients |
| Negative | Failure in | Failure in | Failure in |
| Groups | development | development | development |
| | 6 follicles | 15 follicles | 9 follicles |
| | 4 patients | 9 patients | 5 patients |
| Purpose | Subtracted | Hybridization | Gene |
| | libraries | of the | validation by |
| | Custom- | custom-made | real time |
| | made | Granulosa | PCR |
| | Granulosa | array “B” | |
| | array | Hybridization | |
| | Hybridization | of the | |
| | of the | Affymetrix | |
| | custom-made | Chip “D” | |
| | Granulosa | Gene | |
| | array “A” | validation by | |
| | Hybridization | real time | |
| | of the | PCR | |
| | Affymetrix | | |
| | Chip “C” | | |
| | Gene | | |
| | validation by | | |
| | real time | | |
| | PCR | | |

### RNA Isolation

Total RNA from both mural granulosa cells and cumulus cells was extracted with 1 ml of Trizol reagent (Invitrogen, Burlington, Canada) following the manufacturer's protocol. DNAse treatment was then applied using the DNAse I Amplification Grade kit (Invitrogen, Burlington, Canada) according to the manufacturer's instructions. Extracted RNA was dissolved in 30 μl of water and quantified by spectrophotometry at 260 nm. Total RNA quality and integrity were verified using an Agilent Bioanalyzer 2100 (Agilent Technologies Inc., Santa Clara, USA).

### Microarray Slide Preparation

### Suppressive Subtractive Hybridizations (SSH) for Granulosa Cells and Cumulus Cells

According to the manufacturer's instructions for the BD SMART PCR cDNA Synthesis Kit Clontech, Mountain View, United States), mRNAs from Positive group 1 and Negative group 1 from granulosa and cumulus cells (1 μg) from pools of total RNA were reverse transcribed. The Suppressive Subtractive Hybridization was performed with the PCR Select cDNA Subtraction Kit (Clontech, Mountain View, United States) according to the manufacturer's instructions. DNA amplified previously with the SMART kit from each group of competent mural granulosa and cumulus cells respectively (Positive group 1) served as the tester and non-competent cells for both different group of granulosa and cumulus cells (Negative group 1) as the driver.

cDNA Sequencing

The PCR products were ligated into a vector using the pGEM®-T Easy Vector (Promega, Nepean, Canada) and then transformed into DH5-α-T1 Max Efficiency cells (Invitrogen, Burlington, Canada). For both mural granulosa cells subtracted library and cumulus cells subtracted library, bacterial colonies (1050) were randomly picked and grown in 96-well plates containing 200 μl LB medium (BD Biosciences, Mississauga, Canada) with 50 μg/ml of ampicilin (Sigma-Aldricht, Oakville, Canada). Colonies were incubated at 37° C. with agitation for 6 hours and then kept at 4° C. until PCR amplification of the inserted fragment. For PCR, 2 μl of bacterial suspension were added to a PCR mix containing 1×PCR buffer, 0.25 μM of dNTP, 0.25 mM PCR Nested Primer 1 (5′-AGCGTGGTCGCGGCCGAGGT-3′; SEQ ID NO:1), and 2R (5′-TCGAGCGGCCGCCCGGGCAGGT-3′; SEQ ID NO:2) (Clontech, Mountain View, United States) and 1.25 U of HotMaster Taq DNA Polymerase (Eppendorf, Mississauga, Canada). The PCR conditions consisted of a 94° C. initial denaturating step for 2 minutes and 30 cycles consisting of a denaturating step of 20 seconds at 94° C., an annealing step of 10 seconds at 65° C. and an elongation step of 1 minute at 65° C. and a final step at 65° C. for 7 minutes. PCR product aliquots (3 μl) were visualized on 1% agarose-EtBr gel to verify cDNA length and quality (single band). Amplicons with more than one band were rejected. The remaining bacterial suspension was stored in 20% glycerol at −80° C.

The PCR products were purified and sequenced as described previously. Sequences traces were visualized with the online freeware Chromas 1.45 (http://www.technelysium.com.au/chromas.html) and sequences were loaded into the cDNA Library Manager Program (Genome Canada bioinformatics, Quebec, Canada) trimmed (http://www.phrap.org/phredphrapconsed.html), and compared against the Genbank database (http://www.ncbi.nlm.gov/BLAST/). The BLAST results were compiled into a report chart for each submitted sequence.

Custom-Made cDNA Microarray Preparation

Purified PCR products were speedvac-evaporated (SPD SpeedVac ThermoSavant), suspended in a solution of equal parts of dimethyl sulfoxide (DMSO) and H₂O, and spotted in two replicates in different location on GAPSII glass slides (Corning, Corning, N.Y., United States), using a VersArray Chip WriterPro robot (Bio-Rad, Mississauga, Canada). In addition to human mural granulosa and cumulus cells subtracted libraries, other libraries previously obtained in our lab were also spotted on the slide in two replicates. These libraries are from a bovine cumulus cell subtracted library and a bovine competent granulosa cell subtracted library (Robert C et al. (2001) Biol Reprod 64, 1812-20). A SpotReport Alien and Plant cDNA Array Validation System (Stratagene, Ottawa, Canada) were printed as negative controls. Human actin, tubulin and GAPDH cDNAs acted as positive controls and a fragment of the green fluorescent protein (GFP) was used as an exogenous positive control. DNA was then cross-linked with ultraviolet rays (300 mV) and quality control was performed with Terminal Deoxynucleotidyl Transferase Assay (GE healthcare, Quebec, Canada).

### Microarray Hybridizations

Custom-Made cDNA Slide Hybridizations

Total RNA from Positive and Negative groups 2 of mural granulosa cells were amplified using the RiboAmp™ RNA Amplification kit (Molecular Devices, Mountain View, United States) according to the manufacturer's instructions. Briefly, total RNA was reversed transcribed with a primer incorporating a T7 RNA polymerase promoter sequence. Double-stranded cDNA was synthesized, column-purified (Qiagen, Mississauga, Canada) and used as a template to drive in vitro transcription using the T7 polymerase. This global amplification was linearly amplified by one round and the resulting aaRNA was column purified and the quantity of UTP-amino allyl RNA (aaRNA) was estimated by spectrophotometry at 260 nm. Probes were labelled with Alexa Fluor 555 and 647 reactive dye packs (Invitrogen, Burlington, Canada) according to the protocol from Molecular Probes. Slides were hybridized overnight at 55° C. (for cDNA) or 50° C. (for RNA) with labelled purified probes using the SlideHyb #1 buffer (Ambion, Austin, United States). Hybridizations were performed in an ArrayBooster using the Advacard AC3C (The Gel Company, San Francisco, USA). Slides were then washed twice with 2×SSC/0.5% SDS for 15 minutes at 55° C. (for cDNA) or 50° C. (for RNA) and twice with 0.5×SCC/0.5% SDS for 15 minutes at 55° C. (for cDNA) or 50° C. (for RNA).

Experimental Design for the Custom-Made cDNA Microarray Hybridizations

Two hybridizations were performed using different pools of patients (groups 1 and 2). For the first hybridization, forward-subtracted PCR products from the human mural granulosa cells library (Positive and Negative groups 1) were used as probes to hybridize custom-made cDNA Microarray. For the second hybridization, RNA from both Positive and Negative groups 2 linearly amplified by one round of T7, were used as probes.

Slides were scanned using the VersArray ChipReader System (Bio-Rad, Mississauga, Canada) and analyzed using the ChipReader and ArrayPro Analyzer software (Media Cybernetics, Bethesda, USA). Data analysis was performed as described previously. Fluorescence signal intensities for each replicate were log₂ transformed and normalized by the Loess method, and corrected for background. The determination of the background signal threshold was performed with the SpotReport cDNA controls (Stratagene, Ottawa, Canada), which determine the background (t=m+2×sd, where “t” is the calculated threshold, “m” the mean and “sd” the standard deviation of the negative control data, n=58). Transcripts above the threshold were considered as present in granulosa cells, whereas the other transcripts were eliminated from the analysis. If one replicate was lower than the background, the clone was completely eliminated from the analysis. For both hybridizations, candidates with a log₂ ratio more than 2 were listed and candidates appearing in both lists were Given More Attention.

### Affymetrix Slide Hybridizations

Two Affymetrix human genome arrays were hybridized with the Positive and Negative groups 1 and 2 respectively at the CREMO (Centre de recherche du CHUL, Quebec, Canada). Double stranded cDNA synthesized by reverse transcription was obtained from 250 ng of RNA and amplified twice according to the Affymetrix instructions. Biotin-labelled aRNA was produced from the cDNA from mural granulosa cells and used to probe the Affymetrix human genome array (HG-U133_Plus_{—}2array) (Affymetrix, Lexington, United States) (http://www.affymetrix.com/technology/index.affx). This gene chip contains probes for 33,000 well-substantiated human genes (44,700 transcripts). Hybridizations and washes were performed using the Affymetrix gene chip system according to the manufacturer's instructions. Average difference and expression level of genes were calculated according to absolute and comparison analysis algorithms as recommended by the manufacturer. A ratio more than 2 (Positive groups: Negative groups) was used to select candidates.

### Candidate Gene Selection

Clone selection of clones for further analysis was based on the microarray results from the custom-made cDNA array slides and the Affymetrix slides. A total of 115 different markers were then selected and graded according to their number of occurrences in different libraries, their presence in the human granulosa library, their repetition in the same library, and the signal intensities. After selection and grading, 10 candidate genes were validated by quantitative real time PCR (CYP19A1, CDC42, HSD3B1, SERPINE2, ADX) and 2 housekeeping genes (ACTIN and GAPDH) were used as an internal control.

### Real-Time PCR

Primers of each candidate gene were designed with the Primer3 web interface using sequences derived from NCBI corresponding to our library sequences (Table 2).

Real time analysis measured and compared the three different groups of mural granulosa cells for the Positive and Negative groups with the same procedure already published. Briefly, for each sample, a reverse transcriptase was performed using 50 ng granulosa cells RNA (quantified by spectrophotometer) with the Sensiscript kit (Qiagen, Mississauga, Canada) according to the manufacturer's directions. GFP RNA (7 pg) was added to the RNA mixture as an exogenous control for the reaction. To confirm that the right product was amplified, all amplifications were visualized on an agarose gel (2%) and then sequenced.

TABLE 2
Information and sequences of specific primers used for amplification in
Real Time PCR

| | | | | | | | Fluorescence |
|---|---|---|---|---|---|---|---|
| | | | GenBank | UniGene | Product | Annealing | acquisition |
| | | | accession | accession | size | temperature | temperature |
| Genes | Primers sequences | SEQ NO: | number | number | (bp) | (° C.) | (° C.) |
| CYP19A1 | Up 5′-GCACATCCTCAATACCAGGTC | 3 | NM_000103 | Hs.511367 | 380 | 56 | 84 |
| | Low 5′-TTTGAGGGATTCAGCACAGAC | 4 | | | | | |
| CDC42 | Up 5′-ACGACCGCTGAGTTATCCACAAAC | 5 | NM_001791 | Hs.597524 | 262 | 57 | 82 |
| | Low 5′-ATACTTGACAGCCTTCAGGTCACG | 6 | | | | | |
| HSD3B1 | Up 5′-TGTGCCAGTCTTCATCTACACC | 7 | NM_000198 | Hs.364941 | 101 | 55 | 83 |
| | Low 5′-TGTTTTCCAGAGGCTCTTCTTC | 8 | | | | | |
| SERPINE2 | Up 5′-TGAAGGAGCCGCTGAAAGTTCTTG | 9 | NM_00616 | Hs.38449 | 451 | 59 | 81 |
| | Low 5′-ACCTCCCAGAACAGAAACACTTGC | 10 | | | | | |
| SERPINA3 | Up 5′-ACAAGATGGAGGAAGTGGAAGCCA | 11 | NM_001085 | Hs.534293 | 347 | 59 | 87 |
| | Low 5′-CCTGTTGAAACGCACAATGGTCCT | 12 | | | | | |
| ADX | Up 5′-TCAACCTGTCACCTCATCTTTG | 13 | NM_004109 | Hs.744 | 168 | 57 | 80 |
| | Low 5′-AGGCACTCGAACAGTCATATTG | 14 | | | | | |
| RSG2 | Up 5′-CTGTGACCTGCCATAAAGACTG | 15 | NM_002923 | Hs.78944 | 179 | 57 | 81 |
| | Low 5′-CAGACCACCTATTCCCTTCTTG | 16 | | | | | |
| NRP1 | Up 5′-CCCTGTGGTTTATTCCCAGAAC | 17 | NM_003873 | Hs.131704 | 191 | 56 | 86 |
| | Low 5′-GAGACTTGTGGAGCAAGACACG | 18 | | | | | |
| EGR-1 | Up 5′-GCCATAGGAGAGGAGGGTTC | 19 | NM_001964 | Hs.326035 | 251 | 58 | 82 |
| | Low 5′-GGGTCAGGCATATGATGGAG | 20 | | | | | |
| PGK1 | Up 5′-ACTGTGGTCCTGAAAGCAGCAA | 21 | NM_000291 | Hs.652416 | 449 | 59 | 86 |
| | Low 5′-TTAAGGGTTCCTGGCACTGCAT | 22 | | | | | |

CYP19A1, Homo sapiens Cytochrome P450, family 19, subfamily A, polypeptide 1; CDC42, Homo sapiens Cell division cycle 42; HSD3B1, Homo sapiens Hydroxy-delta-5-steroid dehydrogenase, 3 beta- and steroid delta-isomerase 1; SERPINE2, Homo sapiens Serine (or cysteine) proteinase inhibitor, clade E (nexin, plasminogen activator inhibitor type 1), member 2; SERPINA3, Homo sapiens Serine (or cysteine) proteinase inhibitor, clade A (alpha-1 antiproteinase, antitrypsin), member 3; ADX, Homo sapiens Adrenodoxin; RSG2, Homo sapiens Regulator of G-protein signalling 2; NRP1, Homo sapiens Neuropilin 1; EGR-1, Homo sapiens Early growth response 1; PGK1, Homo sapiens Phosphoglycerate kinase 1.

### Statistical Analysis

Data normalization for each gene in every pool of amplified cDNA was performed by calculation as a ratio to the level of GFP RNA as already published (Data are presented as mean±SEM. GAPDH and β-Actin housekeeping genes were assessed to verify the stability of RNA quantity. The evaluation of mRNA differences between the Positive groups and Negative groups was done by a nonparametric two-tailed t-test. Differences were considered statistically significant at the 95% confidence level (P>0.05).

### Results

### Suppressive Subtractive Hybridization

Using the SSH technique, we subtracted mRNA against reverse transcribed from 6 follicles that resulted in a successful pregnancy (Positive group 1) and 6 follicles leading to an embryo with failure in development (Negative group 1). After cloning and transformation, 1050 clones were selected from each subtracted library, where 852 clones from the human mural granulosa cells subtracted library and 842 clones from the human cumulus cells subtracted library were positive for a single insert (Table 3). Clones were then sequenced and analyzed using the nucleotide-nucleotide BLAST (blastn) program on the NCBI database. In the granulosa and cumulus cell subtracted libraries, 89% and 68% of the total sequences were from genes with known functions respectively, leading to a total of 465 and 645 unique sequences in each library (Table 3). These two subtracted libraries share only 58 unique sequences, which constitute evidence that these two populations of cells possess a repertoire of different potential candidate genes.

TABLE 3
Characteristics of Human subtracted libraries

| Human | Number | Average | | Sequences | Sequences with |
|---|---|---|---|---|---|
| subtracted | of se- | inserts | Unigen | with known | uncharacterized |
| library | quences | size (bp) | target | function | functions |
| Granulosa | 852 | 457 | 645 | 89% | 11% |
| Cumulus | 842 | 416 | 465 | 68% | 32% |

Known sequences, match with a sequence already characterized; Uncharacterized sequences, match with a clone, BAC, RIKEN or hypothetical protein.

### Custom-Made Granulosa Microarray Design

A cDNA microarray of follicular cells ESTs containing 2278 transcripts coding for more than 1200 different genes was made of human and bovine subtracted granulosa and cumulus cells libraries. Preliminary hybridizations demonstrated that human probes can hybridize successfully with both human and bovine cDNA microarrays.

### Microarray Hybridizations

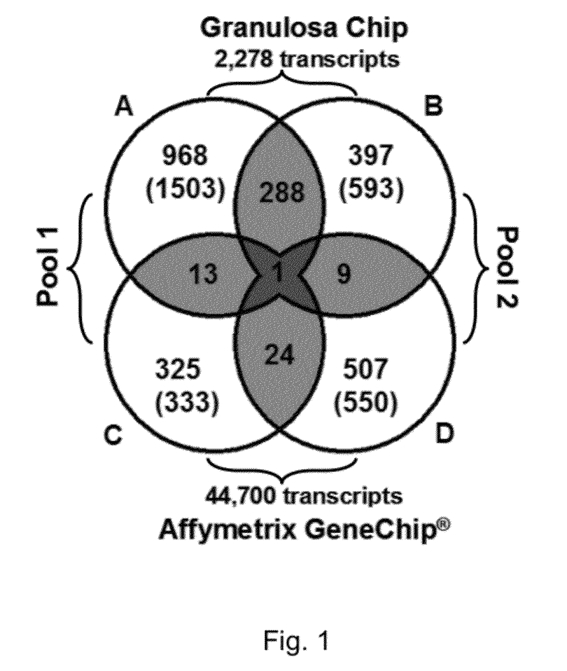

Two hybridization experiments were performed with the Custom-made Granulosa Microarray (FIG. 1). A first hybridization was done with the subtracted samples obtained by the SSH technique. A total of 1503 transcripts of the total 2278 transcripts demonstrated strong ratio for the positive group (potential true positive). In the second hybridization, we used a second pool of mural granulosa cells from different groups of patients (Positive and Negative groups) and obtained 593 transcripts with a strong ratio for the positive group. It is important to note that the material for these 2 hybridizations was obtained through different amplification procedures, SMART-PCR and T-7 amplification respectively, leading to a more stringent group of genes being positive in both hybridizations.

Comparison of the positive clone lists from the two different pools on the Custom-Made Granulosa Microarray resulted in the identification of 288 different genes with a strong intensity (ratio greater than 2) in both hybridizations. It represents 13% of the total transcripts of the Custom-Made Granulosa Microarray.

For the human Affymetrix GeneChip®, a total of 325 and 505 genes had a ratio higher than 2 between positive and negative groups for the two hybridizations respectively. Comparison of the same two pools used for granulosa microarray hybridization on the Affymetrix chip identified 24 genes. The Affymetrix slide contains a vast majority of genes that are not specific to granulosa cells and therefore would not have been positive (0.05% of the 44,700 transcripts). Moreover, comparison of both custom-made microarrays hybridizations and Affymetrix Chips hybridizations shown that 13 genes were in common for the first pool (Group 1) and 9 genes were in common for the second pool (Group 2) with the two different Chips. There was little overlap between the gene lists and CYP19A1 is the only gene that had a strong ratio in all four hybridizations.

### Real Time PCR Candidate Genes Selection

The selection of the competent candidate genes was based on the result of the hybridizations on both platforms. For custom-made microarray hybridizations, a log₂ ratio higher than 2 for the signal intensity was considered as expressed positive. Different parameters were used for the selection of potential candidates. Clones were selected and categorized according to their known functions, their hybridization intensities and their presence in more than one hybridization, their number of occurrences in the same library. Furthermore, we have selected clones with functions known to be involved in oocyte competence.

### Real Time PCR

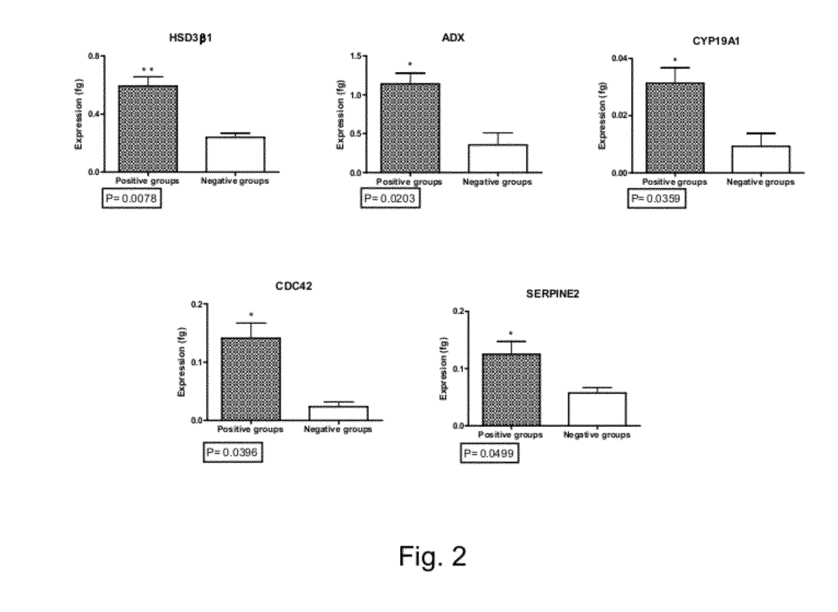

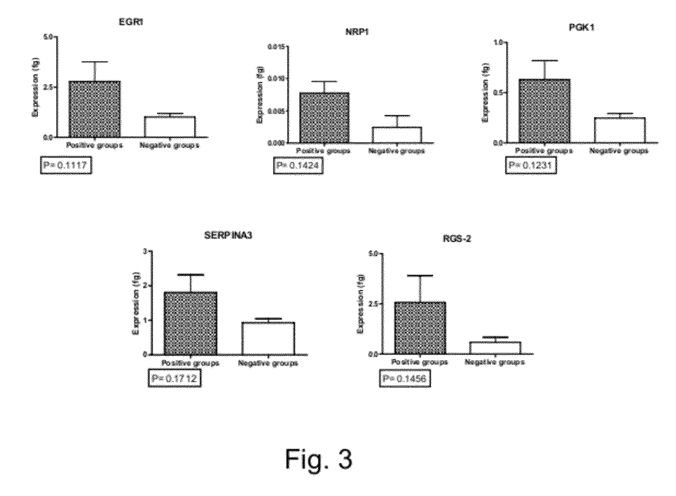

Real time PCR was performed with all 3 pools of human mural granulosa cells from each group (Positive and Negative groups). The expression of housekeeping genes β-Actin and GAPDH was similar (P>0.05) in both groups (Table 5). From the 18 candidate genes selected, five genes (ADX (P=0.0203), CYP19A1 (P=0.0359), cdc42 (P=0.0396), SERPINE2 (P=0.0499) and 3βHSD 1 (P=0.0078) had a statistical difference between competent and non competent cells group (P<0.05) (FIG. 2). The 3βHSD 1 (P=0.0078) had a higher gene expression in the Positive groups (P<0.01) (FIG. 2). Genes such EGR1 (P=0.1117), PGK1 (P=0.1231), NRP-1 (P=0.1424), RGS2 (P=0.1456) and SERPINA3 (P=0.1712) were not statistically different between the two groups, mainly due to larger variation in the levels measured, but could be considered as potential indicators of follicular competence (FIG. 3).

Results presented here have identified 5 potential follicular markers associated with embryo quality resulting in a successful pregnancy in humans. The markers are Adrenodoxin (ADX), Cytochrome P450 aromatase (CYP19A1), Cell division cycle 42 (cdc42), Serpin peptidase inhibitor Glade E member 2 (SERPINE2), and 3-beta-hydroxysteroid dehydrogenase 1 (3βHSD1).

We believe that the markers are likely to originate from mural granulosa cells, although we are aware that with the method of follicular fluid aspiration, it is difficult to obtain a pure sample of granulosa cells. The follicular cells may contain some cumulus cells, blood cells and perhaps some stromal/theca cells, but our protocols was build to reduce the chances of contaminates appearing in the candidate genes. Firstly, the subtractive hybridization should have removed any contaminant clones if present in both the Positive and Negative groups. Secondly, for clinical aspects, the cell population present in the analyzed samples must reflect the biological tissue samples recovered in normal IVF, in that case positive marker could be useful even if not of granulosa cells origin. Lastly, it has been shown that samples with 75% purity were indistinguishable from the pure sample in gene expression profiles using both custom-made arrays and the Affymetrix microarray technology.

This study incorporated two platforms, both custom-made microarrays and the Affymetrix arrays with two different species in order to strengthen the criteria for candidate gene selection. Two different hybridizations were performed with the custom-made microarray, firstly to remove false positives and then with a new pool of RNA. These two hybridizations shared 288 genes with higher expression in the Positive group with competent embryos compared to the Negative group (12,64% of total transcripts on the granulosa microarray). With the Affymetrix Chip, only 24 transcripts (0.05% of the total transcripts on the chip) shown a ratio higher than 2 in both hybridizations. This is low considering that all the genes on the Custom-made granulosa microarray were supposed to be present on the Affymetrix U133 array Chip®, as it represents the human genome. Therefore, it is surprising that only one gene, the CYP19A1, was present in the four different hybridizations across the two chips. Some studies have demonstrated the added value of cross-platform microarrays, which gives more reproducible results, but because platforms are based on different experimental protocols, hybridization and analysis, the comparison from several sources of array may be complicated and unreliable. Our results suggest that, because custom-made granulosa microarrays produced in our laboratory represents a population of genes differentially expressed in granulosa and cumulus cells, this microarray technique has more sensitivity and accuracy to detect minute differences in the gene expression in the same tissue compared to the Affymetrix array.

Following candidate gene selection, expression level of 18 genes was more precisely assessed for their robustness as marker for their possible involvement in follicular competence and 5 (28%) genes were statistically significant between the Positive groups and the Negative groups. These results for the candidate validation are in accordance with similar microarray studies using library subtraction and further validation with quantitative real-time PCR (Fair T (2003) Anim Reprod Sci 78, 203-16.

In this study, three of the genes (ADX, 3βHSD, CYP19A1) that are significantly more expressed in the follicles resulting in a pregnancy are involved in steroidogenesis. Both Adrenodoxin (ADX) and 3-beta-hydroxysteroid dehydrogenase 1 (3βHSD1) are responsible for progesterone synthesis, while cytochrome P450 aromatase (CYP19A1) metabolizes androgen into estradio1-17β in granulosa cells. Adrenodoxin (ADX) is a member of the ferredoxin family and is a component in the electron transfer system for mitochondrial cytochrome P₄₅₀. In mitochondria, pregnenolone is produced from cholesterol by ADX, adrenodoxin transferase and also cytochrome P₄₅₀-side chain cleavage (cytochrome P₄₅₀ scc). Pregnenolone can then be metabolized to progesterone by the 3βHSD in granulosa cells.

Following hCG administration, the mRNA expression of ADX is strongly upregulated in rat granulosa cells to reach maximum expression at 4 hours post treatment. Thereafter, mRNA expression gradually decreases until ovulation (12 hours after hCG) (Espey L L, Richards J S (2002) Biol Reprod 67, 1662-70). Similarly, LH or hCG is the major stimulator of 3βHSD mRNA expression in rat granulosa cells, and like ADX, 3βHSD mRNA expression decreases before ovulation. The expression of 3βHSD in bovine granulosa cells is higher in the dominant follicle than in other subordinate follicles, suggesting that 3βHSD may be associated in the selection mechanism of the dominant follicle. Furthermore, other studies showed that dominant follicles require expression of 3βHSD in human granulosa cells. The P450 aromatase (CYP19A1) is well known to be stimulated by FSH and expressed in high concentrations in dominant follicles. Therefore, higher expression level of these three enzymes appears to be related to hormonal induction (FSH and LH) to the production of steroid hormones (estrogen and progesterone) and possibly to follicular dominance mechanisms.

Level of SERPINE2 (P=0.0499), and cdc42 (P=0.0396) mRNA in human granulosa cells was also correlated with follicles that resulted in a pregnancy. SERPINE2 is a member of a family of protease inhibitors that use a conformational change to inhibit target enzymes. Serpins appear to be ubiquitous and are involved in a multitude of cellular functions, such as apoptosis and chromatin condensation. The expression of SERPINE2 is higher in dominant follicles in the cow, is increased by FSH but decreases after the LH surge. The cdc42 is a member of the Rho family member of GTP-binding proteins involved in many cellular functions. cdc42 can delay the rate of apoptotic progression and then influences programmed cell death.

The majority of genes found to be more expressed (P<0.05) or tended to be more expressed (0.05>P<0.2) in competent follicles are either known to be induced by the LH (hCG) surge (3βHSD, ADX, EGR1, SERPINE2, PGK1, RGS-2, SERPINA3), expressed in the dominant follicle (3βHSD, SERPINE2, CYP19), involved in follicular development (NRP-1) or in anti-apoptotic role (cdc42). However, for some of the genes found to be good indicators of oocyte competence, their expression is reported to be lower at the moment of ovulation (CYP19A1, 3βHSD, ADX (Espey L L, Richards J S (2002) Biol Reprod 67, 1662-70), SERPINE2, EGR1, NRP-1, RGS-2).

It is well known that the luteinization process begins before ovulation. In human IVF, during the ovarian stimulation protocol, the injection of LH/hCG in high concentrations can stimulate an early luteinization of the granulosa cells. In this study, genes selected in granulosa cells from follicles bearing a competent oocyte are known to be induced by LH/hCG. However, some genes, like adrenodoxin (Espey L L, Richards J S (2002) Biol Reprod 67, 1662-70), CYP19A1, SERPINE2, are known to be expressed in early stage of corpus luteum formation. Following the LH surge, competent follicles could be those which have rapidly started the luteinization process.

A second possible hypothesis could be that the dominant follicle gradually acquires LH receptors before the preovulatory LH surge. The LH receptors mRNA expression increases linearly with the increase of follicular diameter. Therefore, in the context of the superovulation protocol, follicles that possess characteristics similar to a dominant follicle would contain more LH receptors. The desensitization of the receptor is achieved by the dissociation of its agonist. However, because hCG/LH has high affinity with the LH receptor, the dissociation of the agonist is considered irreversible. Thus, the association of LH with the receptor, the desensitization, phosphorylation and internalization is an important control of the presence of LH receptors. This process is able to limit the cellular response following activation of the receptor. High concentrations of LH/hCG lead to mass receptor internalization negative feedback and then desensitization of the target cell to LH. Therefore, in the context of the follicular stimulation protocol, the follicle with the highest sensitivity to LH would be the one responding most strongly with increased expression of the LH-inducible genes.

In summary, the microarray approach is a very useful tool for the discovery of new genes and to provide information with respect to oocyte competence. This technology will help to define the transcriptome of granulosa cells associated with a competent oocyte and also improve the selection of healthy oocytes/embryos resulting in good pregnancy rates. The information about genes expressed in competent follicles will also aid the refinement of hormonal treatments in human patients once the mechanism is fully understood.

### Example 2

### Intra Patient Markers in Human Follicular Cells Associated with Competent Oocytes

### Follicular Cells Recovery

Mural granulosa cells were obtained from women (n=40) that were undergoing IVF treatments at the Fertility Center at the Ottawa Hospital, Canada. Women with major indications for IVF, such as tubal infertility, unexplained infertility including endometriosis stage I/II/III and partners not requiring ICSI were recruited to the study. Patients with polycystic ovary syndrome (PCO), or partners with severe male factor requiring ICSI were not included in the study. The procedure was performed with the approval from the Ottawa Hospital Research Ethic Board.

Following ovarian stimulation, follicular fluid, follicular cells and oocytes from individual follicles were collected by ultrasound-guided follicular aspiration using a double lumen needle. The oocytes and surrounding cumulus cells were removed for IVF procedure. The mural granulosa cells recovery was performed as described previously in Example 1. After the recovering procedure, cells were rapidly frozen ant stored in liquid nitrogen until RNA extraction.

Data (fertilization, embryo development, embryo morphology, transfer and pregnancy) generated from each follicle was recorded by an embryologist. From the 40 patients recruited to the study, we have selected patients who produced both follicular cells from follicles that resulted in an embryo with a successful pregnancy (positive samples) and follicular cells that resulted in an embryo arrested in its development before 8 cell stage (negative samples). Samples from 9 patients were in accordance with these criteria. From these 9 patients, a range of 3 to 11 follicles were aspirated for an average of 6.44 follicles per patient, and an average of 3.77 embryos was obtained per woman. Depending of the IVF protocol used, one or two (average of 1.67) embryos were transferred at either day 3 (7 patients) or day 5 (2 patients). A total of 15 positive samples and 9 negative samples served for Q-PCR analysis. Pregnancy was confirmed by the presence of a fetal heartbeat by ultrasound at 6 to 8 weeks for 8 patients and by a biochemical pregnancy for 1 patient.

### RNA Extraction

Total RNA from mural granulosa cells was extracted with 1 ml or Trizol reagent (Invitrogen, Burlington, Canada) following the manufacturer's protocol. RNA was then further purified using the RNeasy total RNA clean-up protocol with the optional DNAse treatment (Qiagen). The concentration and integrity of the RNA samples were assessed spectrophotometrically at 260 nm and on an Agilent Bioanaliser 2100 (Agilent Technologies)) running an aliquot for the RNA samples on the RNA 6000 Nano LabChip (Agilent Technologies). Only RNA that displayed intact 18S and 28S peaks was reverse transcribed to cDNA for real-time PCR analysis.

### Real Time PCR

Primers design and sequences has been described previously in Example 1. Real time analysis measured and compared each samples of mural granulosa cells from an individual follicles (positive and negative samples) with the same procedure already described in the Example 1. β-Actin, Glyceraldehyde-3-phosphate dehydrogenase (GAPDH) and Ubiquitin housekeeping genes were assessed to verify the stability of RNA quantity.

TABLE 4
List of selected genes for intra-patients validation

| | | GenBank | UniGene |
|---|---|---|---|
| | | accession | accession |
| Genes | Genes full name | number | number |
| CYP19A1 | Homo sapiens | NM_000103 | Hs.511367 |
| | Cytochrome P450, family | | |
| | 19, subfamily A, | | |
| | polypeptide 1 | | |
| CDC42 | Homo sapiens Cell | NM_001791 | Hs.597524 |
| | division cycle 42 | | |
| HSD3B1 | Homo sapiens Hydroxy- | NM_000198 | Hs.364941 |
| | delta-5-steroid | | |
| | dehydrogenase, 3 beta- | | |
| | and steroid delta- | | |
| | isomerase 1 | | |
| SERPINE2 | Homo sapiens Serine (or | NM_00616 | Hs.38449 |
| | cysteine) proteinase | | |
| | inhibitor, clade E (nexin, | | |
| | plasminogen activator | | |
| | inhibitor type 1), member | | |
| | 2 | | |
| SERPINA3 | Homo sapiens Serine (or | NM_001085 | Hs.534293 |
| | cysteine) proteinase | | |
| | inhibitor clade A (alpha-1 | | |
| | antiproteinase, | | |
| | antitrypsin), member 3 | | |
| ADX | Homo sapiens | NM_004109 | Hs.744 |
| | Adrenodoxin | | |
| RSG2 | Homo sapiens Regulator | NM_002923 | Hs.78944 |
| | of G-protein signalling 2 | | |
| NRP1 | Homo sapiens Neuropilin | NM_003873 | Hs.131704 |
| | I | | |
| EGR-1 | Homo sapiens Early | NM_001964 | Hs.326035 |
| | growth response 1 | | |
| PGK1 | Homo sapiens | NM_000291 | Hs.652416 |
| | Phosphoglycerate kinase | | |
| | 1 | | |

### Analysis

### Intra Patient Real Time PCR Validation

Analysis of the gene expression stability over the different positive and negative samples was performed using the geNorm software This analysis relies on the principle that the expression ratio of two ideal internal control genes is identical in all samples, regardless of the experimental condition or cell type, and determined as the standard deviation of the logarithmically transformed expression ratios. Using the software, the internal control gene stability (the M value) was calculated as the average pair wise variation of a particular gene with respect to the rest of the genes, and ranking was made based on these values. The most stable reference genes were identified by stepwise exclusions of the least stable gene and recalculating the M values.

This analysis give us the mRNA quantificationin each patient for granulosa cells from individual follicle that produce and embryo associated with a pregnancy outcome or had a failure in embryo development.

TABLE 5
Quantification of mRNA level by real-time PCR in granulosa cells from follicles that
resulted in an embryo transfer and a single pregnancy (Positive embryos) and between granulosa
cells from follicles that produced embryos that arrested in development (Negative embryos).
Results are normalized by a gene normalization factor calculated with the geNorm software.

| | | 3BHSD | CYP19 | ADX | CDC42 | SERPINE2 |
|---|---|---|---|---|---|---|
| PATIENT | | (fg) | (fg) | (fg) | (fg) | (fg) |
| Patient 1 | Positive embryo 1 | 6.917E−06 | 1.398E−07 | 2.625E−06 | 1.745E−07 | 1.887E−07 |
| Patient 1 | Negative embryo | 4.142E−06 | 4.172E−07 | 1.634E−06 | 1.609E−07 | 8.261E−07 |
| Patient 2 | Positive embryo 1 | 1.242E−06 | 1.615E−07 | 1.093E−06 | 1.208E−07 | 3.061E−07 |
| Patient 2 | Negative embryo | 2.584E−06 | 1.427E−06 | 1.049E−06 | 1.225E−07 | 1.666E−07 |
| Patient 3 | Positive embryo 1 | 3.416E−06 | 9.085E−08 | 1.680E−06 | 2.535E−07 | 1.241E−07 |
| Patient 3 | Positive embryo 2 | 3.373E−06 | 1.082E−07 | 2.604E−06 | 1.034E−07 | 1.049E−07 |
| Patient 3 | Negative embryo | 2.375E−06 | 4.988E−08 | 1.225E−06 | 1.706E−07 | 1.440E−07 |
| Patient 4 | Positive embryo 1 | 1.840E−05 | 1.466E−07 | 4.801E−06 | 2.764E−07 | 6.729E−07 |
| Patient 4 | Positive embryo 2 | 1.585E−05 | 3.397E−07 | 2.183E−06 | 7.782E−07 | 5.547E−07 |
| Patient 4 | Negative embryo | 8.882E−06 | 3.826E−06 | 2.062E−06 | 2.450E−07 | 5.041E−07 |
| Patient 5 | Positive embryo 1 | 4.676E−06 | 2.745E−07 | 6.796E−07 | 9.349E−08 | 5.285E−07 |
| Patient 5 | Negative embryo | 6.538E−06 | 2.233E−07 | 8.866E−07 | 9.347E−08 | 1.422E−06 |
| Patient 6 | Positive embryo 1 | 2.181E−06 | 8.423E−08 | 1.135E−06 | 1.066E−07 | 4.062E−08 |
| Patient 6 | Positive embryo 2 | 1.055E−05 | 1.749E−07 | 7.388E−06 | 2.646E−07 | 1.087E−07 |
| Patient 6 | Negative embryo | 2.516E−06 | 1.276E−07 | 2.590E−06 | 1.221E−07 | 8.760E−08 |
| Patient 7 | Positive embryo 1 | 2.194E−06 | 4.725E−08 | 5.153E−07 | 5.683E−08 | 9.486E−08 |
| Patient 7 | Positive embryo 2 | 8.180E−06 | 5.441E−07 | 4.206E−06 | 3.227E−07 | 4.465E−07 |
| Patient 7 | Negative embryo | 4.961E−06 | 1.676E−07 | 1.607E−06 | 1.265E−07 | 1.507E−07 |
| Patient 8 | Positive embryo 1 | 1.659E−06 | 1.458E−07 | 2.048E−07 | 2.716E−07 | 1.455E−07 |
| Patient 8 | Positive embryo 2 | 4.763E−06 | 1.825E−07 | 1.392E−06 | 2.449E−07 | 1.467E−07 |
| Patient 8 | Negative embryo | 3.153E−06 | 3.369E−06 | 1.132E−06 | 1.039E−07 | 3.544E−07 |
| Patient 9 | Positive embryo 1 | 1.255E−05 | 2.520E−07 | 1.663E−06 | 1.684E−07 | 2.040E−07 |
| Patient 9 | Positive embryo 2 | 2.811E−06 | 3.969E−07 | 1.377E−06 | 1.011E−07 | 1.857E−07 |
| Patient 9 | Negative embryo | 1.998E−06 | 2.399E−08 | 2.137E−06 | 1.845E−07 | 3.089E−07 |
| | | SERPINA3 | EGR1 | RGS2 | NRP1 | PGK1 |
| PATIENT | | (fg) | (fg) | (fg) | (fg) | (fg) |
| Patient 1 | Positive embryo 1 | 1.686E−05 | 1.472E−06 | 1.418E−06 | 1.799E−08 | 2.054E−06 |
| Patient 1 | Negative embryo | 1.497E−05 | 3.821E−06 | 1.510E−06 | 1.586E−07 | 1.768E−06 |
| Patient 2 | Positive embryo 1 | 3.014E−05 | 5.209E−06 | 9.205E−07 | 5.176E−08 | 1.417E−06 |
| Patient 2 | Negative embryo | 1.133E−05 | 3.514E−06 | 1.305E−06 | 8.145E−08 | 1.333E−06 |
| Patient 3 | Positive embryo 1 | 1.044E−05 | 3.968E−06 | 1.121E−06 | 1.079E−07 | 1.202E−06 |
| Patient 3 | Positive embryo 2 | 1.924E−05 | 2.612E−06 | 1.241E−06 | 1.101E−07 | 2.136E−06 |
| Patient 3 | Negative embryo | 1.862E−04 | 3.392E−06 | 1.150E−06 | 9.047E−08 | 1.278E−06 |
| Patient 4 | Positive embryo 1 | 3.737E−03 | 2.790E−06 | 5.924E−06 | 0.000E+00 | 2.653E−06 |
| Patient 4 | Positive embryo 2 | 1.400E−05 | 1.671E−05 | 2.418E−05 | 0.000E+00 | 1.808E−06 |
| Patient 4 | Negative embryo | 4.049E−05 | 5.643E−06 | 9.239E−07 | 1.693E−07 | 1.223E−06 |
| Patient 5 | Positive embryo 1 | 2.402E−05 | 4.322E−06 | 1.444E−06 | 9.170E−08 | 1.608E−06 |
| Patient 5 | Negative embryo | 2.664E−06 | 7.599E−06 | 1.005E−06 | 2.499E−08 | 1.492E−06 |
| Patient 6 | Positive embryo 1 | 1.439E−05 | 2.253E−06 | 7.325E−07 | 4.186E−08 | 1.214E−06 |
| Patient 6 | Positive embryo 2 | 2.490E−05 | 5.842E−06 | 1.274E−06 | 5.639E−08 | 1.769E−06 |
| Patient 6 | Negative embryo | 5.791E−05 | 3.261E−06 | 5.399E−07 | 3.691E−08 | 5.600E−07 |
| Patient 7 | Positive embryo 1 | 4.195E−05 | 1.178E−06 | 3.353E−07 | 1.293E−08 | 3.675E−07 |
| Patient 7 | Positive embryo 2 | 4.869E−05 | 5.471E−06 | 1.105E−06 | 5.598E−08 | 1.221E−06 |
| Patient 7 | Negative embryo | 9.148E−05 | 2.438E−06 | 5.952E−07 | 2.147E−08 | 9.288E−07 |
| Patient 8 | Positive embryo 1 | 2.293E−03 | 6.401E−07 | 5.330E−06 | 2.560E−09 | 1.138E−06 |
| Patient 8 | Positive embryo 2 | 1.545E−03 | 1.780E−06 | 1.558E−06 | 4.948E−08 | 7.720E−07 |
| Patient 8 | Negative embryo | 8.705E−05 | 1.864E−06 | 2.453E−07 | 2.194E−08 | 1.314E−07 |
| Patient 9 | Positive embryo 1 | 8.313E−03 | 2.717E−05 | 6.758E−06 | 0.000E+00 | 3.382E−06 |
| Patient 9 | Positive embryo 2 | 3.281E−05 | 2.885E−06 | 6.957E−08 | 4.531E−08 | 4.856E−07 |
| Patient 9 | Negative embryo | 2.688E−04 | 5.310E−06 | 1.062E−06 | 2.505E−08 | 2.043E−06 |

### Infra Patient Ratios

A ratio was calculated between normalized mRNA quantification by real time PCR (Positive embryo/Negative embryo). Ratio 1 and 2 in a same patient is calculated with the positive embryo 1 or 2 respectively.

With these results, we can compare the mRNA level ratio between the positive and negative embryos individually in a same patient.

TABLE 6
Intra-patients ratios calculated from mRNA quantification by real time PCR (Positive
embryo/Negative embryo). Ratio 1 and 2 in a same patient is calculated with the
positive embryo 1 or 2 respectively. Ratios in grey have been rejected by the Principal
components analysis (PCA) for further statistical analysis

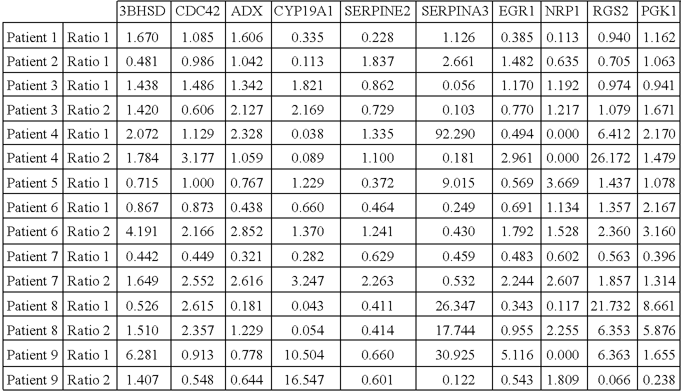

### Principal Components Analysis (PCA) and Paired T-Test Analysis

Six patients had two embryos transferred with one pregnancy outcome. We used the principal components analysis (PCA) to discriminate the true positive and the false positive for the embryos transferred. The PCA analysis use the Euclidian distance. The nearest distance between a positive embryo and a negative embryo from the same patient is qualified false positive and the farther distance is qualified true positive. The false negative data is rejected for the statistical analysis.

This analysis is necessary for further statistical analysis. Following the PCA analysis, we are able to do a paired t-test to determine genes that show a significant difference between true positive embryos and negative embryos.

TABLE 7
Paired t-test statistical analysis for intra patient experiment in
granulosa cells from follicles that resulted in a pregnancy (True
positive embryos) and between granulosa cells from follicles that
produced embryos that arrested in development (Negative embryos).

| Genes | P-Values |
|---|---|
| 3bHSD | 0.2177 |
| ADX | 0.6725 |
| Cdc42 | 0.0991 |
| CYP19A1 | 0.4894 |
| EGR1 | 0.5747 |
| NRP1 | 0.1576 |
| PGK1 | 0.0315 |
| RGS2 | 0.0431 |
| SERPINA3 | 0.4167 |
| SERPINE2 | 0.4194 |

To further explore the link between gene expression an pregnancy, we did a model of conditional logistic regression. This model can predict the cells state in function of the genes expression. Because data are control case type (each woman has a positive cell (case) and a negative cell (control)), we use the conditional logistic regression to study the relation between the expression of genes and the cells state. To do this analysis, we use the exact estimation method.

TABLE 8
Conditional logistic regression

| | Slope | | |
|---|---|---|---|
| Gene | estimation | Odd ratio | p-value |
| 3bHSD | 1.1348 | 3.111 | 0.2227 |
| ADX | 0.3885 | 1.475 | 0.6719 |
| CDC42 | 2.2576 | 9.560 | 0.1055 |
| CYP19A1 | −0.2877 | 0.750 | 0.4961 |
| EGR1 | 0.4575 | 1.580 | 0.5547 |
| NRP1 | −0.5888 | 0.555 | 0.1680 |
| PGK1 | 15.8510* | >999.999 | 0.0039 |
| RGS2 | 2.9068 | 18.299 | 0.0273 |
| SERPINA3 | 0.3106 | 1.364 | 0.4063 |
| SERPINE2 | −0.7633 | 0.466 | 0.4063 |

*median unbiased estimate (we use this estimator when the function of exact conditional probability cannot be maximized. With this method, we can obtain a non biased estimation.

Two genes show a significant relation (p-value<0.05%). For example, we estimate the slope of the relation between the logit of the probability and the log of the expression for the gene RGS2 at 2.91. When the log of the gene expression of RGS2 increase by 1 unit, the logit of the probability increase by 2.91 (the probability vary in the same way than its logit). Furthermore, the odd ratio associated with this analysis, indicate that an increase of the log expression for the RGS2 gene by 1 unit multiply the probability odd to be positive by 18.3.

### Additive Probabilities

With a cut off ratio of 1.5 or 2.0, we did combinations of 1, 2, or 3 genes. The percentage was calculated for each gene with the ratios obtained with true positive and negative embryos. These data show the percentage of patients with a ratio more than 1.5 or 2.0 that are true positive.

With these probabilities, we can have an idea of more appropriate combination of genes to predict a pregnancy.

TABLE 9
Additive probabilities (ratios > 1.5)

| RATIO 1.5 | | | >1 gene | % > 1 | >2 genes |
|---|---|---|---|---|---|
| 3bHSD | SERPINA3 | ADX | 9 | 100 | 4 |
| RGS2 | SERPINA3 | ADX | 9 | 100 | 4 |
| 3bHSD | PGK1 | SERPINA3 | 9 | 100 | 3 |
| cdc42 | SERPINA3 | ADX | 9 | 100 | 3 |
| SERPINA3 | ADX | EGR1 | 9 | 100 | 3 |
| 3bHSD | SERPINA3 | CYP19A1 | 9 | 100 | 2 |
| 3bHSD | RGS2 | SERPINA3 | 8 | 88.89 | 5 |
| 3bHSD | cdc42 | SERPINA3 | 8 | 88.89 | 5 |
| 3bHSD | SERPINA3 | EGR1 | 8 | 88.89 | 4 |
| 3bHSD | SERPINA3 | NRP1 | 8 | 88.89 | 4 |
| PGK1 | SERPINA3 | ADX | 8 | 88.89 | 4 |
| PGK1 | RGS2 | SERPINA3 | 8 | 88.89 | 3 |
| 3bHSD | PGK1 | NRP1 | 8 | 88.89 | 3 |
| PGK1 | cdc42 | SERPINA3 | 8 | 88.89 | 3 |
| PGK1 | SERPINA3 | EGR1 | 8 | 88.89 | 3 |
| RGS2 | SERPINA3 | CYP19A1 | 8 | 88.89 | 3 |
| 3bHSD | PGK1 | SERPINE2 | 8 | 88.89 | 3 |
| 3bHSD | SERPINA3 | SERPINE2 | 8 | 88.89 | 3 |
| cdc42 | SERPINA3 | CYP19A1 | 8 | 88.89 | 3 |
| SERPINA3 | ADX | NRP1 | 8 | 88.89 | 3 |
| SERPINA3 | ADX | CYP19A1 | 8 | 88.89 | 3 |
| RGS2 | ADX | NRP1 | 8 | 88.89 | 2 |
| RGS2 | ADX | SERPINE2 | 8 | 88.89 | 2 |
| SERPINA3 | EGR1 | CYP19A1 | 8 | 88.89 | 2 |
| SERPINA3 | ADX | SERPINE2 | 8 | 88.89 | 2 |
| 3bHSD | SERPINA3 | | 8 | 88.89 | 1 |
| SERPINA3 | ADX | | 8 | 88.89 | 0 |
| 3bHSD | PGK1 | RGS2 | 7 | 77.78 | 5 |
| 3bHSD | PGK1 | cdc42 | 7 | 77.78 | 5 |
| PGK1 | RGS2 | ADX | 7 | 77.78 | 5 |
| cdc42 | RGS2 | SERPINA3 | 7 | 77.78 | 5 |
| RGS2 | SERPINA3 | EGR1 | 7 | 77.78 | 5 |
| cdc42 | SERPINA3 | EGR1 | 7 | 77.78 | 5 |
| RGS2 | SERPINA3 | NRP1 | 7 | 77.78 | 5 |

TABLE 10
Additive probabilities (ratio > 2.0)

| RATIO 2 | | | >1 gene | % > 1 | >2 genes |
|---|---|---|---|---|---|
| cdc42 | SERPINA3 | ADX | 8 | 88.89 | 3 |
| cdc42 | SERPINA3 | CYP19A1 | 8 | 88.89 | 3 |
| RGS2 | SERPINA3 | ADX | 8 | 88.89 | 3 |
| RGS2 | SERPINA3 | CYP19A1 | 8 | 88.89 | 2 |
| SERPINA3 | ADX | EGR1 | 8 | 88.89 | 2 |
| cdc42 | RGS2 | SERPINA3 | 7 | 77.78 | 4 |
| cdc42 | SERPINA3 | EGR1 | 7 | 77.78 | 4 |
| RGS2 | SERPINA3 | EGR1 | 7 | 77.78 | 3 |
| 3bHSD | cdc42 | SERPINA3 | 7 | 77.78 | 3 |
| cdc42 | SERPINA3 | NRP1 | 7 | 77.78 | 3 |
| RGS2 | SERPINA3 | NRP1 | 7 | 77.78 | 3 |
| SERPINA3 | ADX | CYP19A1 | 7 | 77.78 | 3 |
| PGK1 | cdc42 | SERPINA3 | 7 | 77.78 | 2 |
| SERPINA3 | EGR1 | CYP19A1 | 7 | 77.78 | 2 |
| 3bHSD | SERPINA3 | ADX | 7 | 77.78 | 2 |
| PGK1 | SERPINA3 | ADX | 7 | 77.78 | 2 |
| PGK1 | SERPINA3 | EGR1 | 7 | 77.78 | 2 |
| PGK1 | SERPINA3 | CYP19A1 | 7 | 77.78 | 2 |
| cdc42 | SERPINA3 | SERPINE2 | 7 | 77.78 | 2 |
| RGS2 | SERPINA3 | SERPINE2 | 7 | 77.78 | 2 |
| RGS2 | ADX | NRP1 | 7 | 77.78 | 2 |
| RGS2 | NRP1 | CYP19A1 | 7 | 77.78 | 2 |
| SERPINA3 | ADX | NRP1 | 7 | 77.78 | 2 |
| 3bHSD | SERPINA3 | EGR1 | 7 | 77.78 | 1 |
| 3bHSD | SERPINA3 | CYP19A1 | 7 | 77.78 | 1 |
| cdc42 | NRP1 | CYP19A1 | 7 | 77.78 | 1 |
| SERPINA3 | ADX | SERPINE2 | 7 | 77.78 | 1 |
| SERPINA3 | ADX | | 7 | 77.78 | 0 |
| cdc42 | RGS2 | CYP19A1 | 6 | 66.67 | 5 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

We claim:

1. A method for determining competence of a mammalian oocyte, said method comprising assessing expression of at least one granulosa cell marker in granulosa cells of a follicle comprising said oocyte, wherein said granulosa cell marker is selected from the group consisting of CYP19A1, CDC42, DPYSL3, 3βHSD1, EREG, SERPINE2, SCARB1, INHBA, SPRY 2, BACH2, ILST6, ADX, TNFAIP6, SERPINA3, EGR1, NRP1, RGS2, PGK1, and combinations thereof; and wherein said expression level is predicative of oocyte competency.

2. The method of claim 1, wherein said mammalian oocyte is a human oocyte.

3. The method of claim 1, further comprising the step of comparing the expression level of said at least one marker with a control expression level in control granulosa cells.

4. The method of claim 3, wherein a ratio of the expression level of said at least one marker over the control expression level higher than 1.5 is predicative of a better competency.

5. The method of claim 1, wherein assessment of the expression of said at least one granulosa cell marker comprises measuring polynucleotide and/or polypeptide expression levels for said marker.

6. The method of claim 5, comprising measuring DNA and/or RNA levels of a polynucleotide encoding said at least one granulosa cell marker.

7. The method of claim 1, wherein said granulosa cells are obtained by aspiration of follicular fluid before ovulation.

8. The method of claim 1, comprising assessing expression of at least PGK1 or RGS2.

9. The method of claim 1, comprising assessing expression of at least two of said granulosa cell markers.

10. The method of claim 1, comprising assessing expression of at least three of said granulosa cell markers.

11. A method for selecting a mammalian oocyte for in vitro fertilization (IVF) and/or uterus implantation, the method comprising:
obtaining mammalian granulosa cells of a follicle which contains said oocyte;
determining expression level of at least one granulosa cell marker selected from the group consisting of CYP19A1, CDC42, DPYSL3, 3βHSD1, EREG, SERPINE2, SCARB1, INHBA, SPRY 2, BACH2, ILST6, ADX, TNFAIP6, SERPINA3, EGR1, NRP1, RGS2, PGK1, and combinations thereof;
comparing the expression level of said at least one marker with a control expression level in control granulosa cells; and
selecting for IVF and/or for uterus implantation an oocyte which granulosa cells have a higher expression level of said at least one marker when compared with the control expression level.

12. The method of claim 11, wherein said oocyte and granulosa cells are from a human.

13. The method of claim 11, wherein the determination step comprises measuring polynucleotide and/or polypeptide cell levels of said at least one granulosa cell marker.

14. The method of claim 11, comprising measuring DNA and/or RNA levels of a polynucleotide encoding said at least one granulosa cell marker.

15. The method of claim 11, wherein the oocyte for IVF and/or for uterus implantation is selected when a ratio of the expression level of said at least one marker over the control expression level is higher than 1.5.

16. The method of claim 11, wherein said granulosa cell is obtained by aspiration of follicular fluid before ovulation.

17. The method of claim 11, comprising determining expression level of at least one of PGK1 or RGS2.

18. The method of claim 11, comprising determining expression level of at least two of said granulosa cell markers.

19. The method of claim 11, comprising determining expression level of at least three of granulosa cell markers.

20. A method for screening a compound stimulatory or inhibitory to oocyte competence, said method comprising the steps of:
a) treating granulosa cells with a compound to be screened for activity to stimulate or inhibit the competence of an oocyte;
b) determining the expression level of at least one granulosa cell marker selected from the group consisting of CYP19A1, CDC42, DPYSL3, 3βHSD1, EREG, SERPINE2, SCARB1, INHBA, SPRY 2, BACH2, ILST6, ADX, TNFAIP6, SERPINA3, EGR1, NRP1, RGS2, PGK1, and combinations thereof;
c) comparing the expression level measured in step b) with the expression level of non-treated granulosa cells;
wherein a ratio of the expression level of said at least one marker in treated granulosa cells over the expression level of said marker in control granulosa cells higher than 1.5 is indicative of stimulatory effect for said compound whereas a ratio being lower than 1 is indicative of an inhibitory effect.

21. The method of claim 20, wherein said treatment is performed in vitro or in vivo.


SEQUENCE LISTING (22 sequences)

SEQ ID NO: 1 - DNA, 20 bp, Artificial
  Primer
agcgtggtcgcggccgaggt

SEQ ID NO: 2 - DNA, 22 bp, Artificial
  Primer
tcgagcggccgcccgggcaggt

SEQ ID NO: 3 - DNA, 21 bp, Artificial
  Primer
gcacatcctcaataccaggtc

SEQ ID NO: 4 - DNA, 21 bp, Artificial
  Primer
tttgagggattcagcacagac

SEQ ID NO: 5 - DNA, 24 bp, Artificial
  Primer
acgaccgctgagttatccacaaac

SEQ ID NO: 6 - DNA, 24 bp, Artificial
  Primer
atacttgacagccttcaggtcacg

SEQ ID NO: 7 - DNA, 22 bp, Artificial
  Primer
tgtgccagtcttcatctacacc

SEQ ID NO: 8 - DNA, 22 bp, Artificial
  Primer
tgttttccagaggctcttcttc

SEQ ID NO: 9 - DNA, 24 bp, Artificial
  Primer
tgaaggagccgctgaaagttcttg

SEQ ID NO: 10 - DNA, 24 bp, Artificial
  Primer
acctcccagaacagaaacacttgc

SEQ ID NO: 11 - DNA, 24 bp, Artificial
  Primer
acaagatggaggaagtggaagcca

SEQ ID NO: 12 - DNA, 24 bp, Artificial
  Primer
cctgttgaaacgcacaatggtcct

SEQ ID NO: 13 - DNA, 22 bp, Artificial
  Primer
tcaacctgtcacctcatctttg

SEQ ID NO: 14 - DNA, 22 bp, Artificial
  Primer
aggcactcgaacagtcatattg

SEQ ID NO: 15 - DNA, 22 bp, Artificial
  Primer
ctgtgacctgccataaagactg

SEQ ID NO: 16 - DNA, 22 bp, Artificial
  Primer
cagaccacctattcccttcttg

SEQ ID NO: 17 - DNA, 22 bp, Artificial
  Primer
ccctgtggtttattcccagaac

SEQ ID NO: 18 - DNA, 22 bp, Artificial
  Primer
gagacttgtggagcaagacacg

SEQ ID NO: 19 - DNA, 20 bp, Artificial
  Primer
gccataggagaggagggttc

SEQ ID NO: 20 - DNA, 20 bp, Artificial
  Primer
gggtcaggcatatgatggag

SEQ ID NO: 21 - DNA, 22 bp, Artificial
  Primer
actgtggtcctgaaagcagcaa

SEQ ID NO: 22 - DNA, 22 bp, Artificial
  Primer
ttaagggttcctggcactgcat